(19) United States
(12) Patent Application Publication
(10) Pub. No.: US 2009/0326355 A1
(43) Pub. Date: Dec. 31, 2009

(54) Integrated Test System for Monitoring Bodily Fluids

(76) Inventors: Allen J. Brenneman, Goshen, IN (US); Rex J. Kuriger, Danbury, CT (US); Jeffery S. Reynolds, New Fairfield, CT (US); Julian Schlagheck, Munich (DE)

Correspondence Address: NIXON PEABODY LLP, 300 S. Riverside Plaza, 16th Floor, CHICAGO, IL, 60606-6613, US

(21) Appl. No.: 11/990,197

(22) PCT Filed: Aug. 11, 2006
(86) PCT No.: PCT/US06/31453

§ 371 Date: Aug. 21, 2009

Related U.S. Application Data
(60) Provisional application No. 60/707,663, filed on Aug. 12, 2005.

(51) Int. Cl. A61B 5/151

(52) U.S. Cl. 600/347; 600/345; 600/583

(57) ABSTRACT

An integrated diagnostic instrument (10) for analyzing a fluid sample includes a housing (12), a sensor pack (122), a disk drive mechanism (200) and a lancing mechanism (16). The lancing mechanism includes a lance holder (110) adapted to removably engage a base of a lance (86), a plunger (66) coupled to the lance holder, a shaft (70) running through a central portion of the plunger, a spring at least partially surrounding the shaft, and a slider (90) located on a rail on the exterior of the housing.

### FIELD OF THE INVENTION

The present invention relates generally to diagnostic instruments and, more particularly, to an integrated diagnostic instrument for handling multiple sensors that are used in monitoring bodily fluids.

### BACKGROUND OF THE INVENTION

Test sensors (e.g., biosensors) containing reagents are often used in assays for determining the analyte concentration in a fluid sample. The quantitative determination of analytes in body fluids is of great importance in the diagnoses and maintenance of certain physiological abnormalities. For example, lactate, cholesterol, and bilirubin should be monitored in certain individuals. In particular, determining glucose in body fluids is important to diabetic individuals who must frequently check the glucose level in their body fluids to regulate the glucose intake in their diets. Each test requires that a new test sensor be used, and thus, a number of test sensors may be used in a single day.

Cartridges that contain a number of test sensors are used to allow users to carry multiple strips around within a single object. Prior to being used, the sensors typically need to be maintained at an appropriate humidity level so as to insure the integrity of the reagent materials in the sensor. Sensors can be packaged individually in tear-away packages so that they can be maintained at the proper humidity level. As can be appreciated, the opening of these packages can be difficult. Moreover, once the package is opened, the user needs to be sure that the sensor is not damaged or contaminated as it is being placed into the sensor holder and used to test the blood sample. Further, once the sensor is placed in the sensor holder, a fluid sample must be collected and applied to the sensor.

Thus, there exists a need for an integrated diagnostic instrument for storing and dispensing a test sensor while providing a convenient mechanism from collecting and applying a fluid sample to the dispensed sensor.

### SUMMARY OF THE INVENTION

A system and method for analyzing the concentration of an analyte in a fluid sample is disclosed according to one embodiment of the present invention. The system includes a housing, sensor pack, disk drive, and lancet for obtaining and analyzing a fluid sample.

The above summary of the present invention is not intended to represent each embodiment, or every aspect, of the present invention. Additional features and benefits of the present invention are apparent from the detailed description and figures set forth below.

### BRIEF DESCRIPTION OF THE DRAWINGS

FIG. 1 is an upper perspective view of an integrated diagnostic instrument, according to one embodiment of the present invention.

FIG. 2 is a top view of the integrated diagnostic instrument of FIG. 1.

FIG. 3 is a bottom view of the integrated diagnostic instrument of FIG. 1.

FIG. 4 is an upper-perspective side view of the integrated diagnostic instrument of FIG. 1.

FIG. 5a is an upper perspective view of the integrated diagnostic instrument of FIG. 1 with the puller handle in an extended position.

FIG. 5b is an upper perspective view of the integrated diagnostic instrument of FIG. 1 after the puller handle has been moved from the extended position of FIG. 5a to a testing position.

FIG. 6 is an upper perspective view of the integrated diagnostic instrument of FIG. 1 in an open position.

FIG. 7 is an exploded perspective view of a sensor pack used in the integrated diagnostic instrument of FIG. 1, according to one embodiment of the present invention.

FIG. 8 is a lower perspective view of the base portion of the sensor pack of FIG. 7.

FIG. 9 is a side view of the base portion of the sensor pack of FIG. 7.

FIG. 10 is a top view of the base portion of the sensor pack of FIG. 7.

FIG. 11 is an upper perspective view of a test sensor adapted to be enclosed in a sensor cavity of the sensor pack illustrated in FIG. 7, according to one embodiment of the present invention.

FIG. 12 is an exploded perspective view of the component subassemblies of the integrated diagnostic instrument of FIG. 1, according to one embodiment of the present invention.

FIG. 13 is an exploded perspective view of the component parts of an upper case subassembly of the integrated diagnostic instrument for FIG. 1.

FIG. 14 is an exploded perspective view of the component parts of a lower case subassembly of the integrated diagnostic instrument of FIG. 1.

FIG. 15 is an exploded top perspective view of the component parts of a disk drive mechanism and an indexing disk of the integrated diagnostic instrument of FIG. 1.

FIG. 16 is an exploded bottom perspective view of the component parts of a disk drive mechanism and an indexing disk subassembly of the integrated diagnostic instrument of FIG. 1.

FIG. 17 is an exploded perspective view of the component parts of a battery tray subassembly of the integrated diagnostic instrument of FIG. 1.

FIG. 18 is an exploded perspective view of the component parts of an electronics assembly of the integrated diagnostic instrument of FIG. 1.

FIG. 19 is a top perspective view of the electronics subassembly of the integrated diagnostic instrument of FIG. 1.

FIG. 20 is a bottom perspective view of the electronics subassembly of the integrated diagnostic instrument of FIG. 1.

### DESCRIPTION OF ILLUSTRATED EMBODIMENTS

The present invention is directed to an integrated diagnostic instrument for storing and dispensing a plurality of test sensors. The integrated diagnostic instrument in combination with a test sensor may be used to determine concentrations of at least one analyte in a fluid sample on the test sensor. The integrated diagnostic instrument assists a user in collecting a fluid sample, where the fluid sample is, for example, whole blood.

Analytes that may be measured using the present invention include glucose, lipid profiles (e.g., cholesterol, triglycerides, LDL and HDL), microalbumin, hemoglobin A1C, fructose, lactate, bilirubin, or prothrombin. The present invention is not limited, however, to these specific analytes and it is contemplated that other analyte concentrations may be determined. The analytes may be in, for example, a whole blood sample, a blood serum sample, a blood plasma sample, other body fluids like ISF (interstitial fluid) and urine, or other non-body fluid samples.

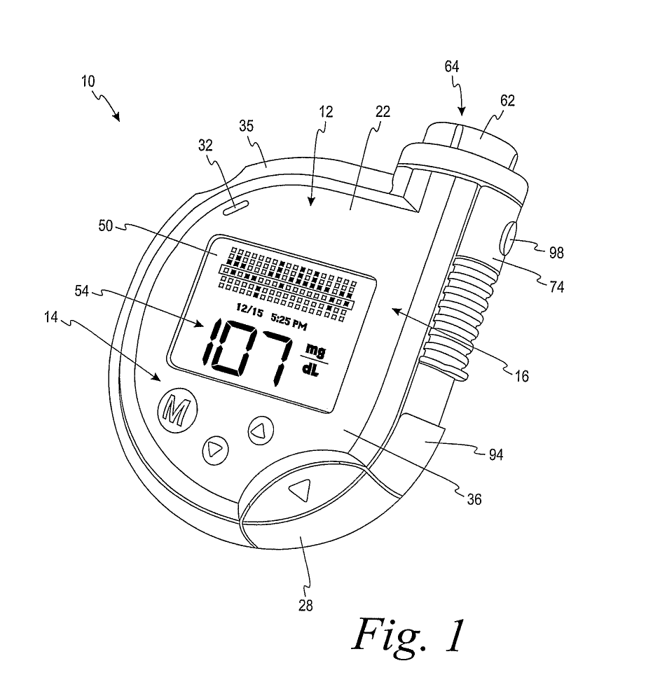

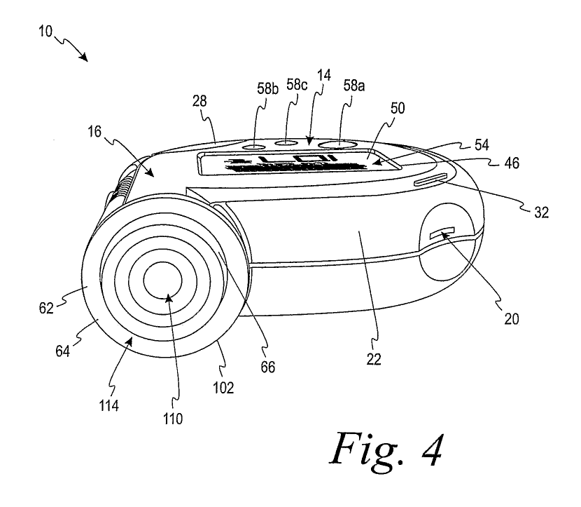

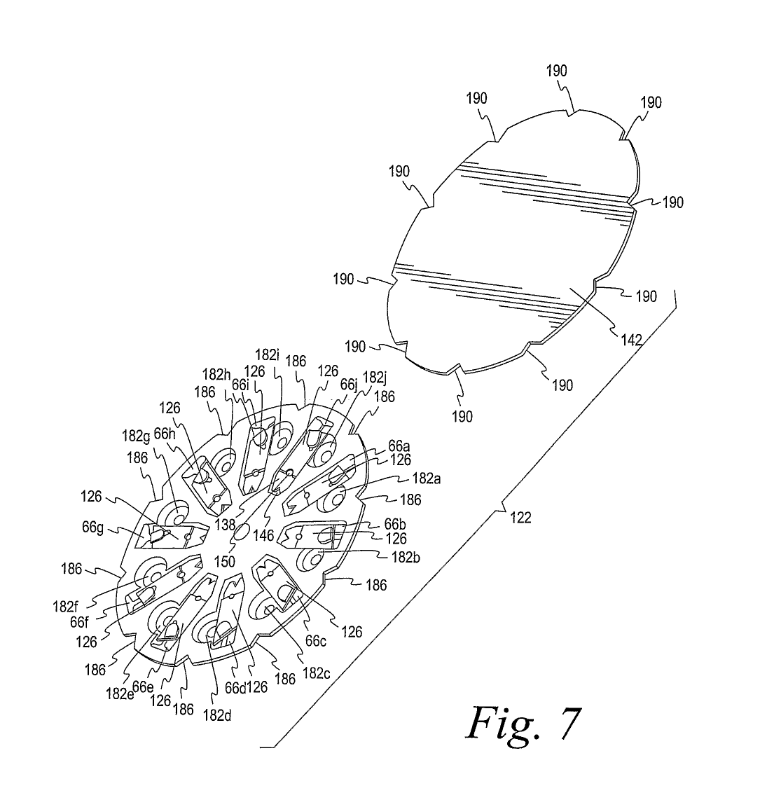

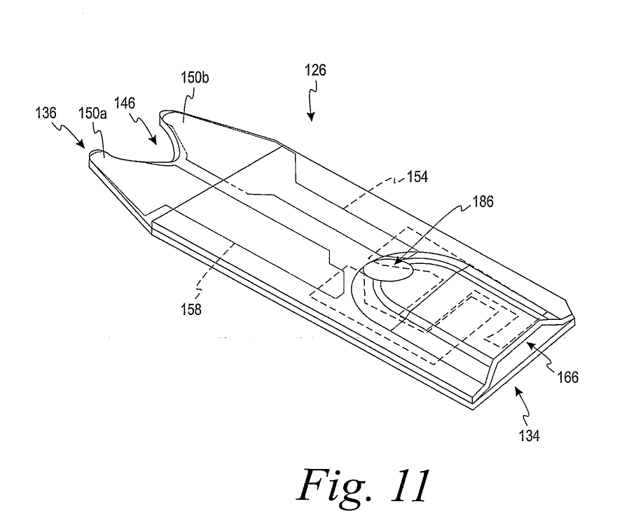

Turning now to the drawings and initially to FIGS. 1-6, an integrated diagnostic instrument 10 is illustrated according to one embodiment of the present invention. The integrated diagnostic instrument 10 comprises a housing 12, a user interface 14, and a lancing mechanism 16. The housing 12 forms at least one test-sensor opening 20 (FIG. 4) therein. The opening 20 is adapted to allow a test sensor 126 (FIG. 11) to be ejected from a sensor pack 122 (FIGS. 7-10) within the housing 12.

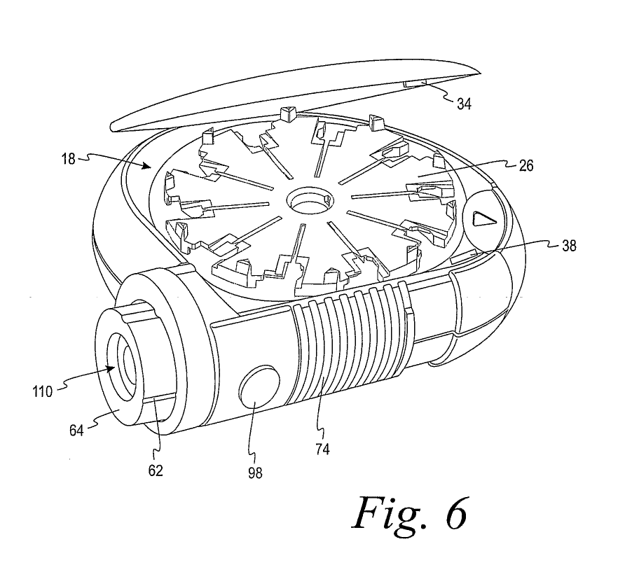

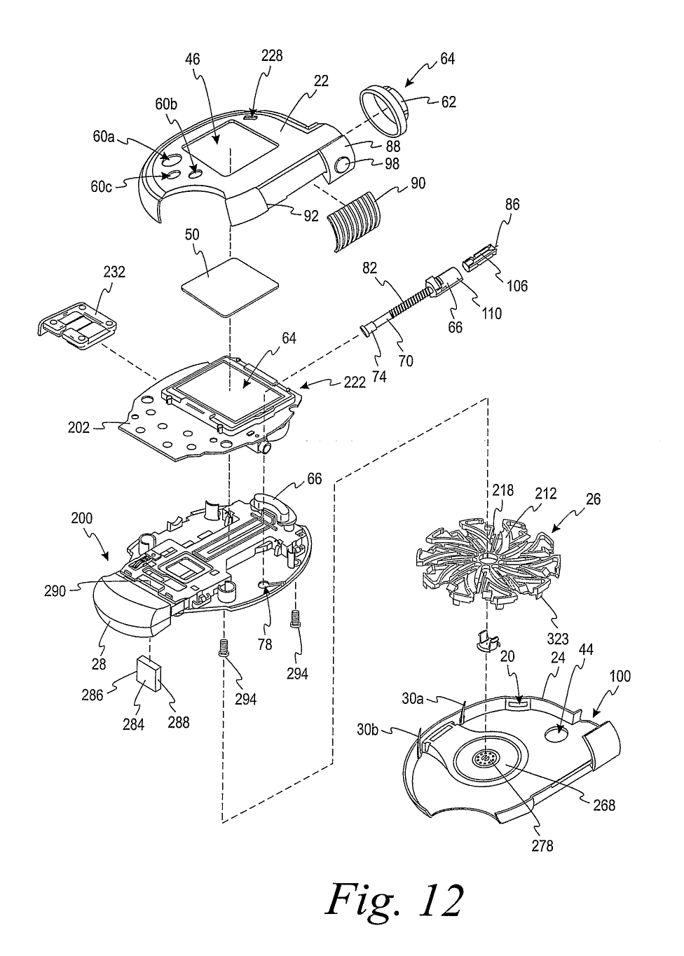

The housing 12 is comprised of an upper case 22 and a lower case 24. The upper case 22 is pivotable with respect to the lower case 24 in a clam-shell fashion so that the sensor pack 122 (FIG. 7) can be positioned on an indexing disk 26 (FIG. 6) within the housing 12. A puller handle 28 is provided within a portion of the housing 12. The puller handle 28, in combination with a disk drive mechanism 200 (FIG. 12), is adapted to allow a user to remove a test sensor 126 from the sensor pack 122.

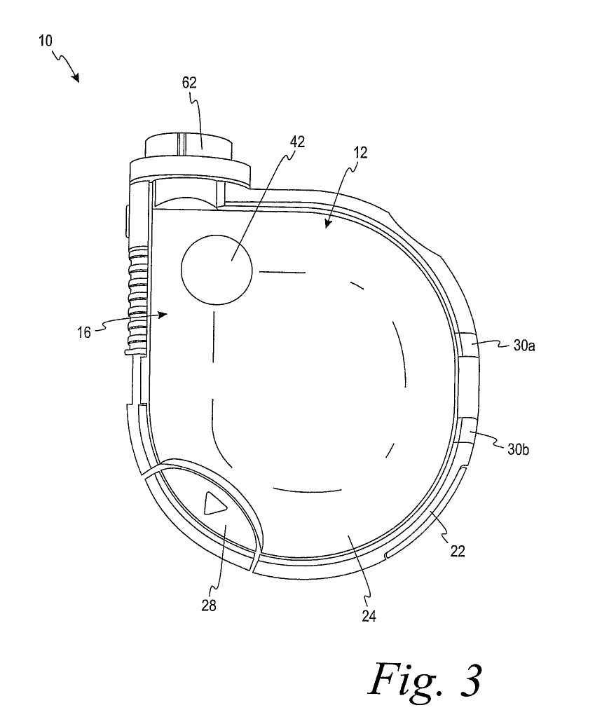

The upper case 22 and the lower case 24 of the instrument are typically made of a polymeric material. Non-limiting examples of polymeric materials include polycarbonate, ABS, nylon, polypropylene, or combinations thereof. The upper case 22 and the lower case 24 are complementary, generally round in shape, hollow containers that are adapted to be pivoted with respect to each other about pivot pins 30a,b (FIG. 3) extending outwardly from the lower case 24 into pivot holes (not shown) in the upper case 22.

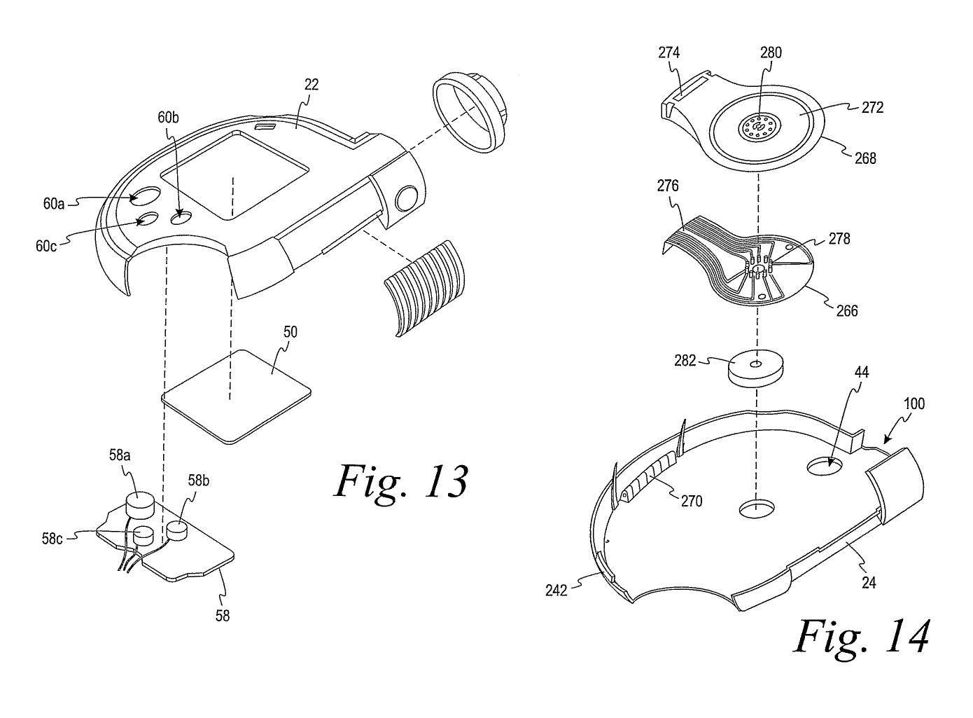

The upper case 22 and lower case 24 are maintained in their closed configuration as shown in FIGS. 1-5 by a latch 34 that is best illustrated in FIG. 6. The latch 34 is located on the upper case 22 and is adapted to engage with a recess 38 formed in the lower case 24. The latch 34 and recess 38 secure the lower case 24 to the upper case 22 when the lower case 24 is moved from an open position (FIG. 6) to a closed position (FIGS. 1-5). To reopen the housing 12, a button 42 is provided that extends through an opening 44 (FIGS. 12-13) formed in the lower casing 24. When the button 42 is depressed in the direction of the housing 12, the latch 34 is disengaged from the recess 38. Upon releasing the button 42 after the latch 34 has been disengaged, the lower case 24 will raise slightly from the upper case 22 and may be fully opened by applying a force to the lower case 24 in the opposite direction of the upper case 22.

As discussed above, the integrated diagnostic instrument 10 includes the user interface 14. The user interface comprises a display unit 54 and a button set 58. As will be more fully described below with respect to FIG. 12, the upper case 22 of the housing 12 forms a generally rectangular opening 46. The opening 46 is adapted to allow a lens 50 to be positioned therein such that a display unit 54 is visible through the lens 50. The display unit 54 is adapted to provide visual information to a user of the integrated diagnostic instrument 10. The display unit 54 is preferably a liquid crystal display (LCD) but any other suitable type of display may be utilized by the present invention. Though the illustrated embodiment shows a generally rectangular opening 46, the opening may be any shape sufficient to allow the display unit 54 (which may also take a variety of shapes) to be visible through the opening.

The user interface 14 also includes a button set 58 that comprises several individual buttons 58a,b,c that extend through a plurality of holes 60a-c (FIGS. 12-13) in the upper case 22 of the housing 12. The individual buttons 58a-c are depressed to operate the electronics of the integrated diagnostic instrument 10. The button set 58 may be used, for example, to recall and have presented on the display 54 the results of prior testing procedures. The button set 58 may also be used to set and display date and time information, and to activate reminder alarms that remind the user to conduct, for example, a blood glucose test according to a predetermined schedule. The button set 58 may also be used to activate certain calibration procedures for the integrated diagnostic instrument 10.

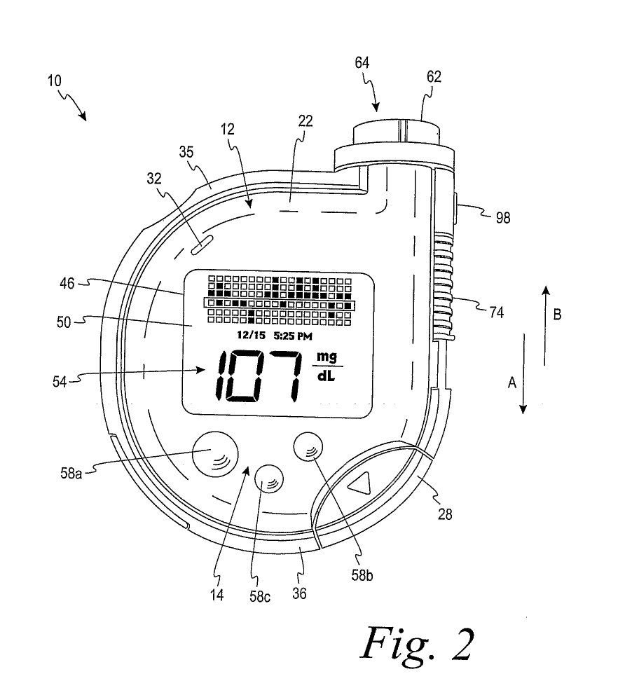

As will be more fully described with respect to FIG. 12, the lancing mechanism 16 of the integrated diagnostic instrument 10 is adapted to assist a user in obtaining a fluid sample. The lancing mechanism 16 includes an endcap 62 that covers a plunger 66 (FIG. 4) for driving a lance 86 (FIG. 12). The endcap 62 has a central aperture (not shown) and protects the test subject from inadvertently contacting the lance 86 positioned therein. A face of the endcap 62 can be touched to the skin of the test subject. The lancing mechanism 16 can then be fired by depressing a firing button 98 (FIGS. 1-2) causing the lance 86 to extend from the endcap 62 and pierce the skin of the test subject.

The lancing mechanism 16 of the integrated diagnostic instrument 10 is adapted to utilize a plurality of lancing endcaps 62. For example, a test subject can attach a standard-site endcap when the test subject prefers to collect a sample from their fingertip. Alternatively, an alternate-site endcap can be attached to the lancing mechanism 16 when an alternate-site test is desired. Typically, an alternate-site endcap is transparent to allow the test subject to look through the endcap to determine the volume of blood that is collected after lancing the skin. The alternate-site endcap may also have a wider opening to allow more skin to insert therein, thus allowing for a deeper lancing of the skin.

The lancing mechanism 16 further includes a slider 90 located on a rail 94 on an exterior portion of the housing 12. The slider 90 is adapted such that movement of the slider 90 in the direction of arrow A (FIG. 2) causes the plunger 66 to move in the direction of arrow A. However, movement of the slider 90 in the direction of arrow B does not cause the plunger to move. A firing button 98 is located on a slider dock 88 and is adapted to actuate the lancing mechanism 16 when the firing button 98 is depressed. The slider 90 is adapted to move along the rail 94 both toward and away from the endcap 62 of the lancing mechanism 16. The rail 94 is formed between the slider dock 88 and a slider stop 92. The rail 94, slider dock 88, and slider stop 92 may be separate components attached to the housing 12 or may be an extension of the housing 12 as illustrated.

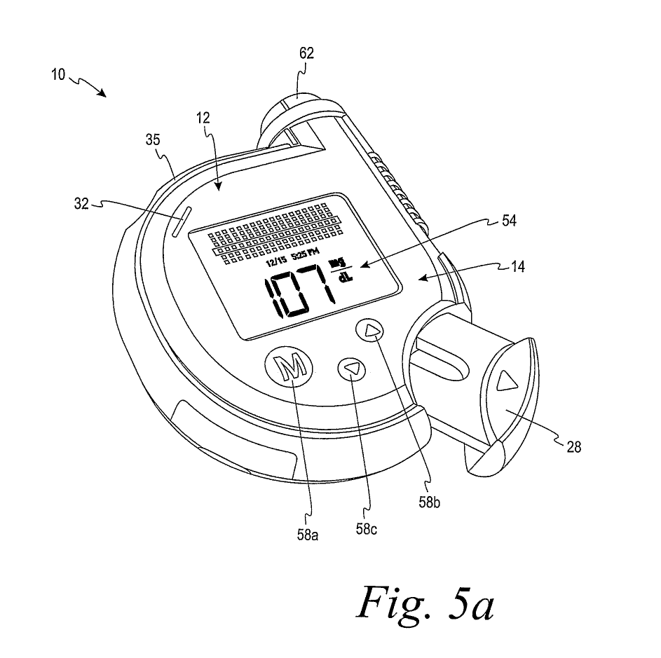

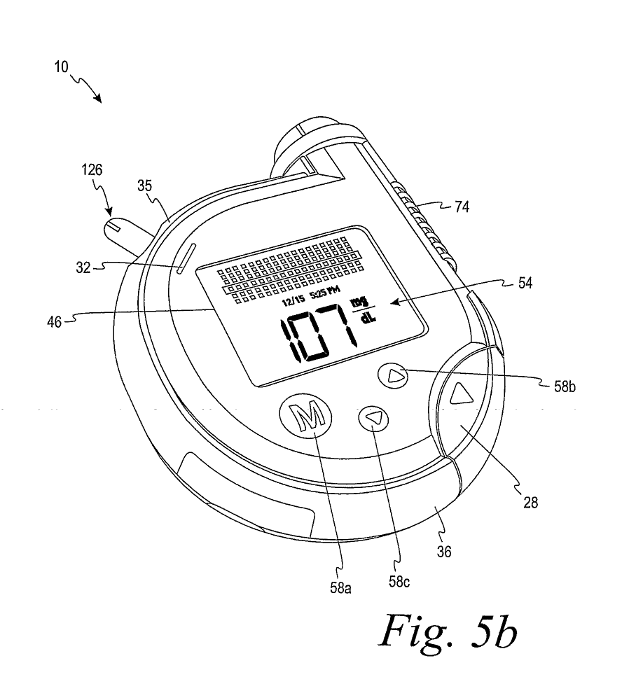

The lancing mechanism 16 is offset from the test-sensor opening 20, as best illustrated if FIGS. 4 and 5b. According to some embodiments of the present invention, the test-sensor opening 20 is at least 20° and less than 180° offset from the lancing mechanism 16. According to some of these embodiments, the test-sensor opening 20 is at least 30° and less than 90° offset from the lancing mechanism 16. According to one embodiment of the present invention, the test-sensor opening 20 is about 45° offset from the lancing mechanism 16, while according to another embodiment, the offset is about 60°. According to still another embodiment of the present invention, the test-sensor opening 20 is about 50° offset from the lancing mechanism 16.

Thus, to obtain and collect a fluid sample (e.g., whole blood) from a test subject, a user (or the test subject) must move the integrated diagnostic instrument 10 from a first position (i.e., a lancing position) to a second position (i.e., a collecting position). According to one method, to move the integrated diagnostic instrument into the first position, the user positions the face 64 of the endcap 62 against the skin of the test subject. The user then depresses the firing button 98 (FIGS. 1-2) to actuate the lancing mechanism 16—piercing the skin of the test subject. The user may then ensure that a sufficient sample size has been obtained from the piercing prior to moving the integrated diagnostic device 10 to the collection position. After piercing the skin of the test subject, the user moves the integrated diagnostic instrument 10 into the second position where a test sensor 126 (FIG. 11)—extending from the test-sensor opening 20—contacts the obtained fluid sample and collects the sample within the test sensor 126 for analysis by the integrated diagnostic instrument. According to the illustrated embodiment (FIGS. 1-6), to move the integrated diagnostic instrument 10 from the first position to the second position, the user rotates the integrated diagnostic instrument 10 about 50°.

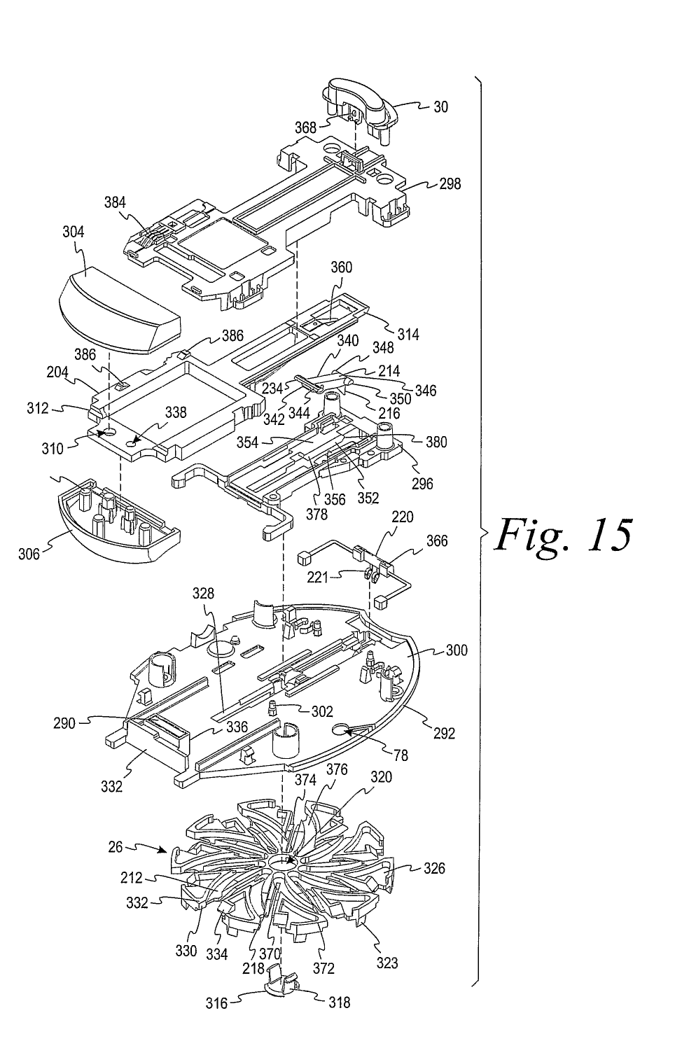

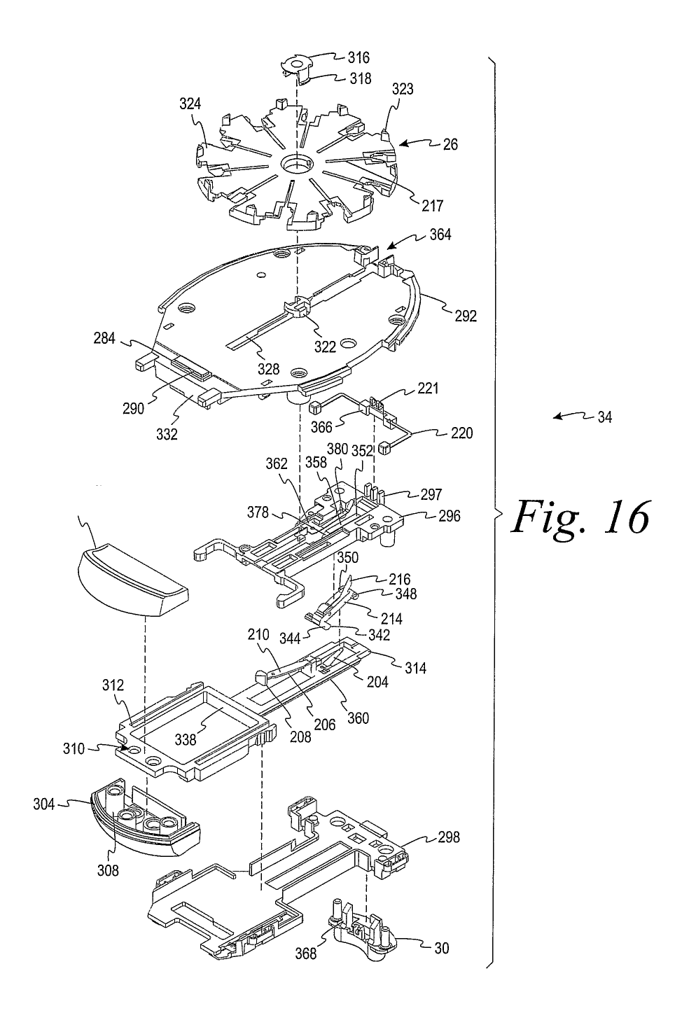

Referring now to FIGS. 7-11, a sensor pack 122 is illustrated according to one embodiment of the present invention. The sensor pack 122 comprises a base portion 140 with a foil 142 sealed thereto. The sensor pack 122 is adapted to house ten sensors 126 with one of the ten sensors 126 in each of the sensor cavities 130a-j. As is illustrated in FIG. 11 each of the sensors 126 has a generally flat, rectangular shape extending from a testing end 134 to a contact end 136. The testing end 134 is angled so that the testing end 134 can puncture an unsevered portion of the foil 142 overlying the sensor cavity 130 as the sensor 126 is being forced out of the sensor cavity 130. The sensor 126 is adapted to be placed into a fluid sample to be analyzed. The contact end 136 of the sensor 126 includes a small notch 146 into which the knife blade 216 (FIGS. 15-16) will become disposed as the knife blade 216 is ejecting the sensor 126 from the sensor cavity 130. The notch 146 provides a target area for the knife blade 216 to contact the sensor 126 and once the knife blade 216 is in contact with the notch 146, the sensor 126 becomes centered on the knife blade 216. Contacts 150a-b near the contact end 136 of the sensor 126 are adapted to mate with metal contact 221 (FIGS. 15-16) on the sensor actuator 220 when the sensor 126 is in a testing position. As a result, the sensor 126 is coupled to the circuitry on the circuit board assembly 202 (FIGS. 12, 18-20) so that information generated in the sensor 126 during testing can be stored and/or analyzed.

Each of the sensors 126 is provided with a capillary channel 166 that extends from the testing end 134 of the sensor 126 to biosensing or reagent material disposed in the sensor 126. When the testing end 134 of the sensor 126 is placed into a fluid sample (for example, blood that is accumulated on a person's finger after the finger has been lanced), a portion of the fluid sample is drawn into the capillary channel 166 by capillary action such that a sufficient amount of fluid required for a test is drawn into the sensor 126. The fluid then chemically reacts with the reagent material in the sensor 126 so that an electrical signal indicative of the analyte concentration in the fluid sample being tested is propagated through the contacts 150a-b (FIG. 11) to the metal contact 221, and thereby through the sensor actuator 220 to the circuit board assembly 202. A vent 168 may be provided along with the capillary channel 166 to facilitate fluid intake into the capillary channel 166 when placed into a fluid sample.

The sensor pack 122 is illustrated as being formed by a generally, circular shaped base portion 140 and the correspondingly configured foil 142, though the sensor pack 122 may, in alternative embodiments, be a variety of shapes (i.e., elliptical, rectangular, triangular, square, etc.) The sensor cavities 130a-j are formed as depressions in the base portion 140 with each of the sensor cavities 130a-j adapted to house one of the sensors 126. As illustrated with respect to the sensor cavity 130a in FIG. 7, each of the sensor cavities 130a-j has a bottom support wall 170 that extends from an inner end 174 to an outer end 178 of the sensor cavity 130a. The support wall 170 is inclined or sloped slightly upward as it extends from the inner end 174 to the outer end 178. This sloping of the support wall 170 results in the sensor 126 being raised slightly as it is being ejected from the sensor cavities 130a-j so that it will avoid or pass above that portion of the heat seal affixing the foil 142 to the base portion 140 along the outer peripheries of the foil 142 and the base portion 140.

Each of the sensor cavities 130a-j is in fluid communication with a corresponding one of the desiccant cavities 182a-j. Each of the desiccant cavities 182a-j is formed by a small depression in the base portion 140 adjacent the corresponding one of the sensor cavities 130a-j. Desiccant material is disposed in the desiccant cavities 182a-j to ensure that the sensor cavities 130a-j are maintained at an appropriate humidity level so that the reagent material in the sensor 126 disposed in the particular sensor cavity 130 is not adversely affected prior to being used. The desiccant material might be in the form of a small bag or round bead of material or any other form that can be readily disposed in the desiccant cavities 182a-j. The amount of such desiccant material placed in each of the desiccant cavities 182a-j will be dependent on the amount that is required to maintain the sensor cavities 130a-j in a desiccated state. One type of desiccant material that could be used is sold under the trademark NATRASORB and is available in powder, pellet and bead forms.

A plurality of notches 186 are formed along the outer peripheral edge of the base portion 140. When the foil 142 is sealed to the base portion 140, a second plurality of notches 190 along the outer peripheral edge of the foil 142 are aligned with the notches 186 on the outer peripheral edge of the base portion 140 to thereby form an integral series of notches along the outer peripheral edge of the sensor pack 122. Each of the notches formed by the notches 186 and 190 is associated with one of the sensor cavities 130a-j in the base portion 140 such that when the sensor pack 122 is mounted on the indexing disk 26 (FIG. 6) with pins 323 (FIGS. 12, 15-16) disposed in the notches 186 and 190, the sensor cavities 130a-j will each be in proper alignment with an individual one of the radially extending grooves 218 (FIGS. 12, 15) in the indexing disk 26.

The foil 142 is adapted to cover the top of the base portion 140 and be affixed to the base portion 140 by heat sealing substantially the entire outer peripheral edge of the foil 142 to the outer peripheral edge of the base portion 140. The foil 142 also is heat sealed about substantially the entire perimeter of each set of the sensor retaining cavities 130a-j and the desiccant cavities 182a-j to seal the sensor retaining cavities 130a-j and the desiccant cavities 182a-j such that the individual sensors 126 are maintained in a desiccated state and isolated from each other. As a result, the opening of one of the sensor cavities 130a-j will not affect the desiccated state of any of the other sensor cavities 130a-j. The foil 142 may be made of any material that will adequately seal the sensor cavities 130a-j and the desiccant cavities 182a-j while providing a material that will can be really severed by the knife blade 216 (FIGS. 15-16) and pierced by the sensor 126 as it is being pushed out from the sensor cavities 130a-j. One type of foil that can be used for the foil 142 is AL-191-01 foil distributed by Alusuisse Flexible Packaging, Inc.

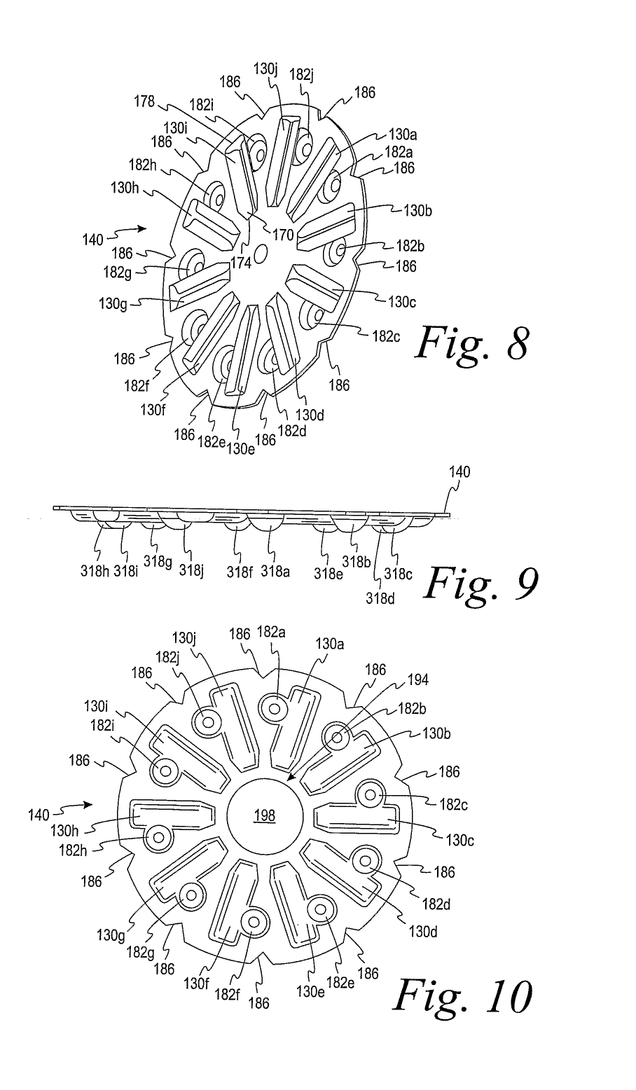

As illustrated in FIG. 10, the base portion 140 includes a label area 194—on the upper, central portion of the base portion 140—inwardly of the sensor cavities 130a-j. A conductive label 198 may be positioned in this label area 194 to provide calibration and production information that may be sensed by calibration circuitry that may be incorporated into the circuit board assembly.

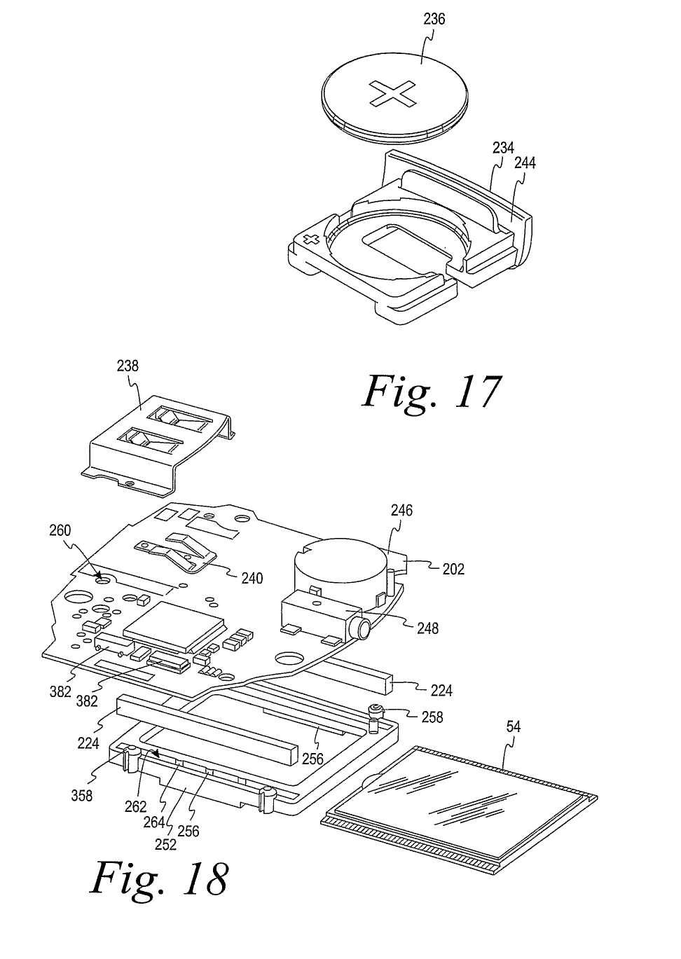

Referring now to FIGS. 12-20, the configuration of the components contained within the housing 12 are illustrated, according to one embodiment of the present invention. The puller handle 28 can be moved to engage a disk drive mechanism, generally designated by the numeral 200 (FIG. 12). To operate the integrated diagnostic instrument 10, the puller handle 28 is first manually pulled from a standby position (FIG. 1) adjacent the rear end 36 of the housing 12 to an extended position (FIG. 5a) away from the rear end 36 of the housing 12. The outward movement of the puller handle 28 causes a disk drive mechanism 200 to rotate the sensor pack 122 and place the next sensor 126 in a standby position prior to being loaded into a testing position (FIG. 5b). The outward movement of the puller handle 28 also causes the integrated diagnostic instrument 10 to turn ON (i.e., the electronic circuitry on the circuit board assembly 202 is activated).

It should be noted that the disk drive mechanism 200 is independent from the operation of the lancing mechanism 16. Thus, if necessary to collect a sufficient fluid sample, multiple punctures can be made to the skin of a test subject using the lancing mechanism 16 without the need to eject another test sensor 126 (FIG. 11) from the sensor pack 122 (FIGS. 7-10) or to discard the previously ejected test sensor 126.

As will be described in greater detail below, the disk drive mechanism 200 includes a disk drive pusher 204 on which an indexing disk drive arm 206 is mounted (see FIGS. 15-16). The indexing disk drive arm 206 comprises a cam button 208 disposed at the end of a plate spring 210. The cam button 208 is configured to travel in one of a plurality of curvilinearly extending grooves 212 on the upper surface of the indexing disk 26. As the puller handle 28 is manually pulled from a standby position adjacent the rear end 36 of the housing 12 to an extended position away from the rear end 36 of the housing 12, the disk drive pusher 204 is pulled laterally towards the rear end 36 of the housing 12. This causes the cam button 208 on the indexing disk drive arm 206 to travel along one of the curvilinearly extending grooves 212 so as to rotate the indexing disk 26. The rotation of the indexing disk 26 causes the sensor pack 122 to be rotated so that the next one of the sensor cavities 130a-j is placed in a ready position.

The puller handle 28 is then manually pushed inwardly from the extended position (FIG. 5a) back to the standby position (FIG. 1). The puller handle 28 can then be pushed slightly more towards the testing end 35 of the housing to place the integrated diagnostic instrument into a testing position (FIG. 5b). In the testing position a portion of a test sensor 126 extends from the test-sensor opening 20 formed in the housing 12. The inward movement of the puller handle 28 causes the disk drive mechanism 200 to remove a sensor 126 from the sensor pack 122 and place the sensor 126 into a testing position on the testing end 35 of the housing 12.

As will be described in greater detail below, the disk drive mechanism 200 includes a knife blade assembly 214 that is pivotally mounted to the disk drive pusher 204 (see FIGS. 15 and 16). As the puller handle 28 is manually pushed from the extended position to the testing position, the disk drive pusher 204 is pushed towards the testing end 35 of the housing 12. This causes the knife blade assembly 214 to pivot downwardly so that a knife blade 216 on the end of the knife blade assembly 214 pierces a portion of the foil 142 covering one of the sensor cavities 130a-j and engages the sensor 126 disposed in one of the sensor cavities 130a-j. As the disk drive pusher 204 continues to move towards the testing end 20 of the upper case 22, the knife blade assembly 214 forces the sensor 126 out of one of the sensor cavities 130a-j and into a testing position at the testing end 35 of the housing 12.

While the disk drive pusher 204 is being pushed from the extended position to the testing position, the cam button 208 on the indexing disk drive arm 206 travels along one of the radially extending grooves 218 to prevent the indexing disk 26 from rotating. Similarly, while the disk drive pusher 204 is being pulled from the standby position to the extended position, the knife blade assembly 214 is in a retracted position so as to not interfere with the rotation of the indexing disk 26.

After a sensor 126 has been completely ejected from one of the sensor cavities 130a-j and pushed into a testing position projecting out from the testing end 35 of the housing 12, the disk drive pusher 204 engages and forces a sensor actuator 220 against the sensor 126 to thereby maintain the sensor 126 in the testing position. The sensor actuator 220 engages the sensor 126 when the puller handle 28 is pushed into the testing position. The sensor actuator 220 couples the sensor 126 to an electronics assembly 222 disposed in the upper case 22. The electronics assembly 222 includes a microprocessor or the like for processing and/or storing data generated during the blood glucose test procedure, and displaying the data on the display unit 54 in the integrated diagnostic instrument 10.

The upper case 22 contains an opening 228 for the button release 32, which projects upwardly through the upper case 22. Once the blood analyzing test is completed, the button release 32 on the upper case 22 is depressed so as to disengage the sensor actuator 220 and release the sensor 126. Depressing the button release 32 causes the disk drive pusher 204 and the puller handle 28 to move from the testing position back to the standby position. At this point, the user can turn the integrated diagnostic instrument 10 OFF or allow the integrated diagnostic instrument 10 to automatically turn OFF pursuant a timer on the electronics assembly 222.

As seen in FIGS. 1-5 and 12-13 the upper case 22 includes a rectangular opening 46 through which a display unit 54 is visible below. The display unit 54 is visible through a lens 50 that is affixed to upper surface of the upper case 22. The display unit 54 is a component of the electronics assembly 222, and is coupled to the circuit board assembly 202 via elastomeric connectors 224 (see FIG. 18). The display unit 54 displays information from the testing procedure and/or in response to signals input by the button set 58 on the upper case 22. For example, the button set 58 can be utilized to recall and view the results of prior testing procedures on the display unit 54. As best seen in FIG. 13, the button set 58 is attached to the upper case 22 from below so that the individual buttons 58a-c project upwardly through button openings 226 in the upper case 22. Each button 58a-c is electrically connected to the circuit board assembly 202 when that particular button 58a-c is depressed.

The upper case 22 also contains a battery opening 230 (FIGS. 5a-b) for a battery tray assembly 232. The battery tray assembly 232 includes a battery tray 234 in which at least one battery 236 is disposed. The battery tray assembly 232 is inserted into the battery opening 230 in the side of the upper case 22. When so inserted, the battery 236 engages battery contacts 238 and 240 on the circuit board assembly 202 so as to provide power for the electronics within the instrument 10, including the circuitry on the circuit board assembly 202 and the display unit 54. A tab 242 on the lower case 24 is configured to engage a slot 244 in the battery tray assembly 232 so as to prevent the battery tray assembly 232 from being removed from the integrated diagnostic instrument 10 when the upper case 22 and the lower case 24 are in the closed configuration.

The electronics assembly 222 is affixed to the upper inside surface of the upper case 22. As best seen in FIGS. 18-20, the electronics assembly 222 comprises a circuit board assembly 202 on which various electronics and electrical components are attached. A positive battery contact 238 and a negative battery contact 240 are disposed on the bottom surface 246 (which is the upwardly facing surface as viewed in FIGS. 18 and 20) of the circuit board assembly 202. The battery contacts 238 and 240 are configured to electrically connect with the battery 236 when the battery tray assembly 232 is inserted into the housing 12. The bottom surface 246 of the circuit board assembly 202 also includes a communication interface 248. The communication interface 248 permits the transfer of testing or calibration information between the integrated diagnostic instrument 10 and another device, such as a personal computer, through standard cable connectors (not shown). In the preferred embodiment shown, the communication interface 248 is a standard serial connector. However, the communication interface 248 could alternatively be an infra-red emitter/detector port, a telephone jack, or radio frequency transmitter/receiver port. Other electronics and electrical devices, such as memory chips for storing glucose test results or ROM chips for carrying out programs are likewise included on the bottom surface 246 and an upper surface 250 of the circuit board assembly 202.

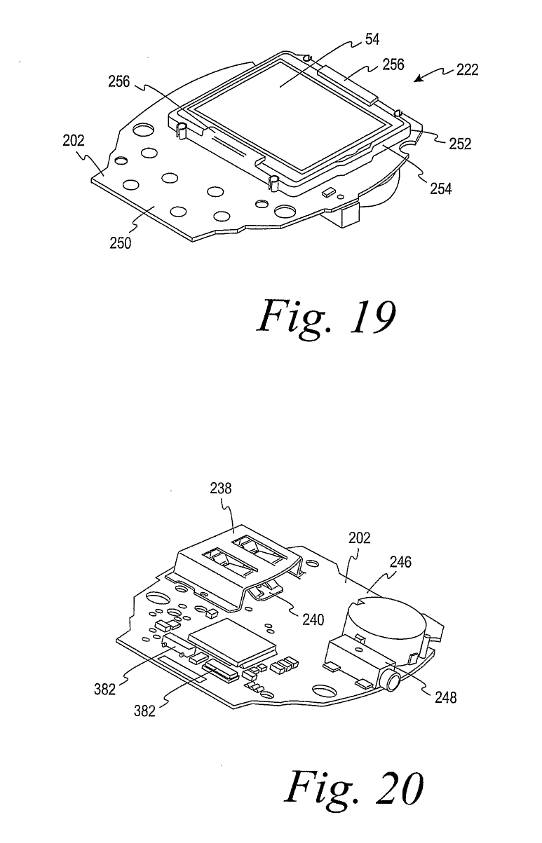

A display unit 54 is affixed to the upper surface 250 (upwardly facing surface in FIG. 19) of the circuit board assembly 202. The display unit 54 is held by a snap-in display frame 252. The snap-in display frame 252 includes side walls 254 that surround and position the display unit 54. An overhang 256 on two of the side walls 254 holds the display unit 54 in the snap-in display frame 252. The snap-in display frame 252 includes a plurality of snap fasteners 258 that are configured to engage mating holes 260 on the circuit board assembly 202. The display unit 54 is electrically connected to the electronics on the circuit board assembly 202 by a pair of elastomeric connectors 224 disposed in slots 262 in the snap-in display holder 252. The elastomeric connectors 224 generally comprise alternating layers of flexible conductive and insulating materials so as to create a somewhat flexible electrical connector. In the preferred embodiment shown, the slots 262 contain a plurality of slot bumps 264 that engage the sides of the elastomeric connectors 224 to prevent them from falling out of the slots 262 during assembly.

The snap-in display frame 252 eliminates the screw-type fasteners and metal compression frames that are typically used to assemble and attach a display unit 54 to an electronic device. In addition, the snap-in display frame 252 also permits the display unit 54 to be tested prior to assembling the display unit 54 to the circuit board assembly 202. The snap-in display frame 252 is more fully described in U.S. Pat. No. 6,661,647 entitled Snap-in Display Frame, which is incorporated herein in its entirety.

The button set 58 also mates to the upper surface 250 of the circuit board assembly 202. As mentioned above, the button set 58 comprises several individual buttons 58a-c that are depressed to operate the electronics of the integrated diagnostic instrument 10. For example, the button set 58 can be utilized to activate the testing procedure of the integrated diagnostic instrument 10. The button set 58 can also be used to recall and have displayed on the display unit 54 the results of prior testing procedures. The button set 58 can also be utilized to set and display date and time information, and to activate reminder alarms which remind the user to conduct a blood glucose test according to a predetermined schedule. The button set 58 can also be used to activate certain calibration procedures for the integrated diagnostic instrument 10.

The electronics assembly 222 further comprises a pair of surface contacts 382 on the bottom surface 246 of the circuit board assembly 202 (see FIGS. 18 and 20). The surface contacts 382 are configured so as to be contacted by one or more fingers 384 on the cover mechanism 298, which in turn are configured to be engaged by a pair of ramp contacts 386 on the disk drive pusher 204 (see FIG. 15). Movement of the puller handle 28 causes the ramp contacts 386 to push the fingers 384 into contact with one or both of the surface contacts 382 so as to communicate the position of the puller handle 28 to the electronics assembly 222. In particular, movement of the puller handle 28 from the standby or testing positions to the extended position will turn the sensor dispensing instrument ON. In addition, if the housing 12 is opened while the puller handle 28 is in the extended position, an alarm will be activated to warn the user that the knife blade 216 may be in the extended position.

It should be noted that the design and configuration of the electronics assembly 222 permits the assembly and testing of the electronics and electrical components prior to assembly of the electronics assembly 222 to the upper case 22 of the integrated diagnostic instrument 10. In particular, the display unit 54, the button set 58, the battery contacts 238 and 240, and the other electronics and electrical components can each be assembled to the circuit board assembly 202 and tested to verify that these components, and the electrical connections to these components, are working properly. Any problem or malfunction identified by the testing can then be corrected, or the malfunctioning component can be discarded, prior to assembling the electronics assembly 222 to the upper case 22 of the integrated diagnostic instrument 10.

The lancing mechanism 16 is affixed to the upper case 22 of the housing 12. The housing 12 has a plunger opening 100 (FIGS. 12-13) formed in both the upper case 22 and the lower case 24. An endcap 62 is removeably attached to the housing 12 at the plunger opening 100. The plunger 66 is adapted to reciprocally move from inside the housing 12 to outside the housing 12 and back through the plunger opening 100. The plunger 66 has a hollow core (not shown) that is adapted to allow the plunger 66 to move along a shaft 70 running through a central portion of the plunger 66. The shaft 70 includes an end portion 74 that is adapted to fit into a slot 78 located on the guide block 292. The slot 78 secures the shaft 70 to the guide block 292 such that movement of the slider 90 will cause the plunger 66 to move along the shaft 70 while the shaft 70 remains motionless. The shaft 70 is at least partially surrounded by the spring 82 that is located between the plunger 66 and the end portion 74 of the shaft 70.

The lancing mechanism 16 is adapted to utilize a lance 86 to pierce the skin of a test subject. The lance 86 is embedded in a plastic base 106 that is removably attached to a lance holder 110 disposed within the endcap 62. The base 106 is removably attached to the lance holder 110 so that the lance 86 can be detached and discarded after use. The opposite end of the lance holder 110 is coupled to the plunger 66. Thus, movement of the plunger 66 by the slider 90 moves the lance holder 110 which, in turn, drives the lance 86.

As mentioned above, the integrated diagnostic instrument 10 may include calibration circuitry for determining calibration and production information about the sensor pack 122. As best seen in FIG. 14, the calibration circuitry comprises a flex circuit 266 located in the lower case 24. The flex circuit 266 is held in position in the lower case 24 by an autocal disk 268 that is connected to the lower case 24 by a pair of pins 270. The autocal disk 268 has a raised central portion 272 configured to engage the sensor pack 122 and hold the sensor pack 122 against the indexing disk 26 when the integrated diagnostic instrument 10 is closed. The autocal disk 268 also has an open area 274 located between the pins 270 to expose contacts 276 on the flex circuit 266.

The flex circuit 266 comprises a plurality of probes 278 that extend upwardly from the flex circuit 266 through holes 280 in the inner region of the autocal disk 268. These probes 278 are connected to the contacts 276 on the end of the flex circuit 266. When the integrated diagnostic instrument 10 is closed with the lower case 24 latched to the upper case 22, the probes 278 make contact with the conductive label 198 on the sensor pack 122 being used in the integrated diagnostic instrument 10. A foam pad 282 is positioned below the flex circuit 266 to provide a biasing force to assure that the probes 278 press against the conductive label 198 with a force sufficient to make an electrical connection. The foam pad 282 also provides a cushioning force so that the probes 278 can move independently with respect to each other as the sensor pack 122 is being rotated by the indexing disk 26. As a result, information, such as calibration and production data, contained on the conductive label 198 can be transmitted via the probes 278 to the flex circuit 266, which in turn couples the data to the electronic circuitry on the circuit board assembly 202 via an elastomeric connector 284. This information can then be used by the electronics assembly 222 to calibrate the integrated diagnostic instrument 10, or can be displayed on the display unit 54.

As best seen in FIG. 12, the elastomeric connector 284 is made of layers of silicon rubber extending from a top edge 286 to a bottom edge 288 with alternate layers having conductive materials dispersed therein to connect contacts on the top edge 286 to contacts on the bottom edge 288. When the upper case 22 and the lower case 24 are closed, the elastomeric connector 284 is compressed in the direction between the edges 286 and 288 such that the contacts along the top edge 286 engage electronic circuitry on the circuit board assembly 202 in the upper case 22, and the contacts along the bottom edge 288 engage the contacts 276 on the flex circuit 266 in the lower case 24. With the elastomeric connector 284 so compressed, low voltage signals can be readily transmitted between the circuit board assembly 202 and the flex circuit 266 through the elastomeric connector 284.

The elastomeric connector 284 is held in position by a slotted housing 290 on the guide block 292. In the preferred embodiment shown, the slotted housing 290 has a serpentine cross-section configured to allow the connector 284 to compress when the upper case 22 and the lower case 24 are closed, while still holding the elastomeric connector 284 when the upper case 22 and the lower case 24 are open. Alternatively, the slotted housing 290 may include inwardly projecting ridges that engage the sides of the connector 284.

The disk drive mechanism 200 is affixed to the upper inside surface of the upper case 22. As best seen in FIG. 12, the disk drive mechanism 200 is attached to the upper case by a plurality of mounting screws 294 that engage posts (not shown) on the upper inside surface of the upper case 22. The mounting screws 294 also pass through and secure the electronics assembly 222 and the lancing mechanism 16, which are disposed between the disk drive mechanism 200 and the upper case 22.

Although the disk drive mechanism 200 will be described in greater detail below, it should be noted that the disk drive mechanism 200 is configured so as to permit the assembly and testing of its operation prior to mounting the disk drive mechanism 200 to the upper inside surface of the upper case 22. In other words, the disk drive mechanism 200 has a modular design that can be tested prior to final assembly of the integrated diagnostic instrument 10.

As best seen in FIGS. 15 and 16, the disk drive mechanism 200 comprises a guide block 292, a sensor actuator 220, a housing guide 296, a disk drive pusher 204, an indexing disk drive arm 206, a knife blade assembly 214, a puller handle 28, a cover mechanism 298, and a button release 32. The housing guide 296 is fixed to the upper surface 300 (as viewed in FIG. 13) of the guide block 292 by one or more pins 302. The disk drive pusher 204 is supported on the housing guide 296 and the guide block 292 in such a manner as to permit the disk drive pusher 204 to slide laterally relative to the housing guide 296 and the guide block 292. The knife blade assembly 214 is pivotally connected to the underside of the disk drive pusher 204, and is guided by the housing guide 296 and the guide block 292. The indexing disk drive arm 206 is also connected to the disk drive pusher 204, and is partially guided by the guide block 292. The puller handle 28 comprises an upper puller handle 304 and a lower puller handle 306 connected to each other by snap-press fittings 308 that pass through holes 310 in the rear end 312 of the disk drive pusher 204. In the preferred embodiment shown, the upper puller handle 304 and the lower puller handle 306 each have a concaved, textured outer surface (i.e., the top and bottom surfaces of the puller handle 28) to facilitate gripping the puller handle 28 between the thumb and finger of a user's hand. The cover mechanism 298 is affixed to the guide block 292 with the disk drive pusher 204 and the housing guide 296 disposed therebetween. The sensor actuator 220 is attached to the guide block 292 and is engaged by the testing end 314 of the disk drive pusher 204 when the disk drive pusher 204 is in the testing position. The button release 32 is slidably connected to the cover mechanism 298 so as to engage the testing end 314 of the disk drive pusher 204 when the disk drive pusher 204 is in the testing position.

In addition, an indexing disk 26 is rotatably secured to the disk drive mechanism 200 by a retainer disk 316 connected through the indexing disk 26 and into guide block 292. As best seen in FIG. 16, the retainer disk 316 has a pair of latch arms 318 that extend through a central hole 320 in the indexing disk 26 and latch into an opening 322 in the guide block 292. The indexing disk 26 includes a plurality of pins 323 protruding from the lower surface 324 thereof. These pins 323 are configured to engage notches 186,190 on the sensor pack 122 (see FIG. 7) so as to align and rotate the sensor pack 122 in accordance with the position of the indexing disk 26. Hence, the pins 323 and the notches 186,190 have the dual purpose of (i) retaining the sensor pack 122 on the indexing disk 26 so that the sensor pack 122 will rotate with the indexing disk 26 and (ii) positioning the sensor pack 122 in proper circumferential alignment relative to the indexing disk 26.

As previously indicated, the disk drive pusher 204 is pulled away from the rear end 36 of the housing 12 (and away from the testing end 35) by a user manually exerting a pulling force on the puller handle 28 to move the handle 28 from the standby position to the extended position. As the puller handle 28 is pulled away from the rear end 36 of the housing 12, the disk drive pusher 204 is guided towards the rear end 36 by the guide block 292, the housing guide 296, and the cover mechanism 298. As the disk drive pusher 204 slides back towards the rear end 36 of the housing 12, the indexing disk drive arm 206 causes the indexing disk 26 to rotate.

The indexing disk drive arm 206 extends rearwardly from the disk drive pusher 204. The indexing disk drive arm 206 includes a plate spring 210 made of spring type material, such as, for example, stainless steel, so as to bias the arm 206 outwardly from the disk drive pusher 204. A cam button 208 is affixed to the distal end of the arm 206, and is configured to engage the upper surface 326 (as viewed in FIG. 15) of the indexing disk 26. In particular, the indexing disk drive arm 206 is bent so as to protrude downwardly through a slot 328 in the guide block 292 such that the cam button 208 projects outwardly from the surface thereof. The slot 328 is designed such that the indexing disk drive arm 206 and the cam button 208 can move along the slot 328 as the disk drive pusher 204 is moved back and forth during the testing procedure. The slot 328 also prevents the indexing disk drive arm 206 from moving sideways with respect to the disk drive pusher 204 (i.e., it provides lateral support to the indexing disk drive arm 206).

As best seen in FIG. 15, the upper surface 326 of the indexing disk 26 comprises a series of curvilinearly extending grooves 212 and a plurality of radially extending grooves 218. The cam button 208 is configured to ride along these grooves 212 and 218 during the movement of the disk drive pusher 204. As the disk drive pusher 204 slides towards the rear end 36 of the housing 12, the cam button 208 moves along one of the curvilinearly extending grooves 212. This causes the indexing disk 26 to rotate. In the preferred embodiment shown, there are ten radially extending grooves 218 and ten curvilinearly extending grooves 212 equally spaced about the circumference of the indexing disk 26, with each radially extending groove 218 being disposed between a pair of curvilinearly extending grooves 212. Accordingly, the movement of the disk drive pusher 204 towards the rear end 22 on the upper case 22 results in a one-tenth rotation of the indexing disk 26.

As the puller handle 28 is pulled away from the rear end 36 of the housing 12 to a fully extended position, the cam button 208 passes over an outer step 330 that separates the outer end 332 of the curvilinearly extending groove 212 from the adjacent radially extending groove 218. The outer step 330 is formed by the difference in depth between the outer end 332 of the curvilinearly extending groove 212 and the outer end 334 of the adjacent radially extending groove 218. In particular, the outer end 334 of the radially extending groove 218 is deeper than the outer end 332 of the curvilinearly extending groove 212. Thus, when the cam button 208 moves from the curvilinearly extending groove 212 into the adjacent radially extending groove 218, the biasing force of the plate spring 210 of the indexing disk drive arm 206 causes the cam button 208 to travel downwardly past an outer step 330. The outer step 330 prevents the cam button 208 from re-entering an outer end 332 of the curvilinearly extending groove 212 when the direction of travel of the disk drive pusher 204 is reversed (as will be explained below).

Rotation of the indexing disk 26 causes the sensor pack 122 to likewise rotate so that the next available sensor cavity 130 is placed in a standby position adjacent to the testing end 35 of the housing 12. The sensor pack 122 rotates with the indexing disk 26 because of the engagement of the notches 186,190 on the sensor pack 122 by the pins 323 on the indexing disk 26. As explained above, each sensor cavity 130 contains a disposable sensor 126 that is used during the fluid sample testing procedure.

Further rearward movement of the disk drive pusher 204 is prevented by a rear wall 336 on the guide block 292. In the preferred embodiment shown, the rear wall 336 includes a slotted housing 290 for holding the elastomeric connector 284 that connects the electronics assembly 222 to the flex circuit 266 disposed in the lower case 24. An interior edge 338 of the disk drive pusher 204 engages the rear wall 336 on the guide block 292 when the disk drive pusher 204 is in the fully extended position (see FIG. 5a).

From the fully extended position, the puller handle 28 is then manually pushed inwardly into a testing position (FIG. 5b). As previously indicated, the inward movement of the puller handle 28 causes the disk drive mechanism 200 to dispense a sensor 126 from the sensor pack 122 and place the sensor 126 into a testing position.

As best seen in FIGS. 15-16, the disk drive mechanism 200 includes a knife blade assembly 214 that is pivotally mounted to the disk drive pusher 204. The knife blade assembly 214 comprises a swing arm 340 having a first end 342 that is pivotally connected to the disk drive pusher 204 by a pair of pivot pins 344. A knife blade 216 is connected to the second end 346 of the swing arm 340. The second end 346 of the swing arm 340 also includes a first cam follower 348 and a second cam follower 350, each in the shape of a transversely extending post. The first cam follower 348 is configured to follow a pathway formed on one side of the knife blade assembly 214 by the guide block 292, the housing guide 296, and the cover mechanism 298. In particular, this pathway is formed by a cam projection 352 on the housing guide 296 that forms an upper pathway 354 between the cam projection 352 and the cover mechanism 298 and a lower pathway 356 between the cam projection 352 and the guide block 292. When the first cam follower 348 is disposed in the upper pathway 354, the knife blade 216 is in the retracted position. On the other hand, when the first cam follower 348 is disposed in the lower pathway 356, then the knife blade 216 is in the extended position. The upper pathway 354 and the lower pathway 356 are connected together at both ends of the cam projection 352 so as to form a continuous loop about which the first cam follower 348 can travel.

The second cam follower 350 engages a cam spring 358 attached to the housing guide 296. As will be explained below, the cam spring 358 guides the knife blade assembly 214 from the lower pathway 356 to the upper pathway 354 when the disk drive pusher 204 is initially pulled rearward from the standby position towards the extended position. The disk drive pusher 204 also comprises a spring 360 for biasing the knife blade 216 towards the extended position when the disk drive pusher 204 is initially pushed forward from the extended position towards the testing position. In the preferred embodiment shown, the spring 360 is a plate spring that presses against the upper side of the swing arm 340.

As the puller handle 28 is manually pushed from the extended position to the testing position, the disk drive pusher 204 is pushed laterally towards the testing end 35 of the housing 12. As the disk drive pusher 204 begins to move forward, the spring 360 biases the swing aim 340 downwardly towards the indexing disk 26 so that the first cam follower 348 engages a sloped surface 362 on the interior end 378 of the cam projection 352 and is forced into the lower pathway 356. This causes the knife blade 216 to assume an extended position whereby the knife blade 216 projects outwardly through a knife slot 217 in the indexing disk 26 to pierce the protective foil 142 covering one of the sensor cavities 130a-j and engage the notch 146 on the contact end 136 of the sensor 126 contained therein. As the disk drive pusher 204 continues to move towards the testing end 35 of the housing 12, the first cam follower 348 continues along the lower pathway 356, thereby causing the knife blade 216 to remain in the extended position projecting through the knife slot 217 so that it will travel along the knife slot 217 and push the sensor 126 forward out of the sensor cavity 130, partially through the test-sensor opening 20, and into a testing position at the testing end 35 of the housing 12. The sensor 126 is in the testing position when the testing end 134 of the sensor 126 projects out of the sensor opening 364 formed on the testing end of the guide block 292 and through the test-sensor opening 20 formed in the housing 12. While in the testing position, the sensor 126 is prevented from being pushed back through the sensor opening 364 by the engagement of the knife blade 216 against the notch 146 on the contact end 136 of the sensor 126.

As the disk drive pusher 204 reaches the testing position, the testing end 314 of the disk drive pusher 204 simultaneously engages the sensor actuator 220 and the button release 32. In particular, the testing end 314 of the disk drive pusher 204 engages and pushes the button release 32 outwardly so as to project upwardly from the upper surface of the upper case 22. At the same time, the testing end 314 of the disk drive pusher 204 engages a contact pad 366 on the sensor actuator 220 so as to force the sensor actuator 220 downward. This downward motion causes a pair of metal contacts 221 on the sensor actuator 220 to project into the sensor opening 364 on the guide block 292 and engage the contacts 150a-b on the sensor 126 for the fluid sample testing procedure. The metal contacts 221 also apply a frictional force to the sensor 126 so that the sensor 126 does not prematurely fall out of the sensor openings 364 and 20 prior to completion of the testing procedure. In the preferred embodiment shown, the metal contacts 221 are somewhat flexible and are made of stainless steel. The housing guide 296 includes support ribs 297 disposed adjacent to the metal contacts 221 so as to prevent the metal contacts 221 from bending. The metal contacts 221 permit the transmission of electrical signals between the sensor 126 and the electronics assembly 222 during the glucose testing procedure.

When the fluid sample testing procedure is complete, the button release 32 is depressed to release the sensor 126 from the testing position. The button release 32 has a sloped contact surface 368 that engages the testing end 314 of the disk drive pusher 204 at an angle. As the button release 32 is depressed, the sloped contact surface 368 slides along the testing end 314 of the disk drive pusher 204, thereby causing the disk drive pusher 204 to move rearward from the testing position and into the standby position. The movement of the disk drive pusher 204 to the standby position also causes the testing end 314 of the disk drive pusher 204 to disengage from the contact pad 366 on the sensor actuator 220, thereby allowing the sensor actuator 220 to move away from and disengage the sensor 126. The sensor 126 can then be removed by tipping the testing end 35 of the integrated diagnostic instrument 10 downwardly or by grasping the sensor 126 and applying a pulling force away from the integrated diagnostic instrument 10.

As mentioned above, when the disk drive pusher 204 is pushed from the extended position towards the testing position, the cam button 208 on the indexing disk drive arm 206 travels along one of the radially extending grooves 218 to prevent the indexing disk 26 and the sensor pack 122 from rotating. The radially extending groove 218 includes a sloped portion 370 that changes the depth of the groove 218. In particular, the sloped portion 370 decreases the depth of the radially extending groove 218 so that the middle portion of the radially extending groove 218 is shallower than the curvilinearly extending grooves 212. The radially extending groove 218 also comprises an inner step 372 near its inner end 374 (i.e., near the center of the indexing disk 26). The inner step 372 is formed along the juncture of the inner end 374 of the radially extending groove 218 and an inner end 376 of the curvilinearly extending groove 212. As the disk drive pusher 204 is pushed from the extended position towards the testing position, the cam button 208 travels up the sloped portion 370 of the radially extending groove 218, past the inner step 372, and into the adjacent curvilinearly extending groove 212. The biasing force of the plate spring 210 of the indexing disk drive arm 206 causes the cam button 208 to travel downwardly past the inner step 372. The inner step 372 prevents the cam button 208 from re-entering the radially extending groove 218 when the direction of travel of the disk drive pusher 204 is reversed (as explained above in connection with the outward movement of the disk drive pusher 204).

As the disk drive pusher 204 reaches the testing position, the first cam follower 348 passes the exterior end 380 of the cam projection 352. At the same time, the second cam follower 350 passes over the end of the cam spring 358, which retracts upwardly and out of the way as the first cam follower 348 nears the exterior end 380 of the cam projection 352. Once the first cam follower 348 has passed the end of the cam spring 358, the cam spring 358 moves downwardly so as to engage and guide the second cam follower 350 upwardly when the direction of travel of the disk drive pusher 204 is reversed and pulled outward towards the extended position. In particular, when the disk drive pusher 204 is subsequently pulled outward towards the extended position, the cam spring 358 guides the second cam follower 350 upwardly so that the first cam follower 348 enters the upper pathway 354 and the knife blade 216 is retracted.

The disk drive pusher 204 is pulled outwardly to initiate the testing procedure. During the outward motion of the disk drive pusher 204, the cam button 208 on the indexing disk drive arm 206 travels along one of the curvilinearly extending grooves 212 so as to rotate the indexing disk 26. During this outward motion, the first cam follower 348 on the knife blade assembly 214 travels along the upper pathway 354. As a result, the knife blade 216 is retracted from the knife slot 217 on the indexing disk 26 so that the indexing disk 26 is free to rotate in response to the action of the cam button 208 in the curvilinearly extending groove 212. As the disk drive pusher 204 reaches the fully extended position, the first cam follower 348 passes the interior end 378 of the cam projection 352 and is guided into the lower pathway 356 by the biasing force of the spring 360 on the swing arm 340 of the knife blade assembly 214.

Prior to operating the integrated diagnostic instrument 10, a sensor pack 122 must first be loaded into the integrated diagnostic instrument 10 if one has not already been so loaded, or if all of the sensors 126 in the previously loaded sensor pack 122 have been used. To load a sensor pack 122, the lower case 24 and the upper case 22 are opened by depressing the latch 388 on the lower case 24. In the preferred embodiment shown, the opening of the lower case 24 and the upper case 22 causes the elastomeric connector 284 to separate from the contacts 276 on the autocal disk 268, thereby breaking the electrical connection between the autocal disk 268 and the electronics assembly 222. This causes an electronic counter (which is part of the electronics assembly 222) that keeps count of the number of unused sensors 126 in the sensor pack 122 to re-set to zero (0).

The opened housing 12 is then turned so that the lower surface 324 of the indexing disk 26 faces upwardly as shown in FIG. 6. A sensor pack 122 is then placed on the indexing disk 26 by aligning the notches 186,190 along the periphery of the sensor pack 122 with the pins 323 on the indexing disk 26. The lower case 24 is then pivoted on to the upper case 22 so as to enclose the sensor pack 122 within the housing. Once the lower case 24 is secured to the upper case 22 by the latch 388, the integrated diagnostic instrument 10 is ready for operation.

The following is a brief description of the operation of the integrated diagnostic instrument 10. First, the puller handle 28 is manually pulled from a standby position (FIG. 1) adjacent the rear end 36 of the housing 12 to an extended position (FIG. 5a) away from the rear end 36 of the housing 12. The outward movement of the puller handle 28 causes the integrated diagnostic instrument 10 to turn ON. The outward movement of the puller handle 28 also causes the cam button 208 on the indexing disk drive arm 206 to travel along one of the curvilinearly extending grooves 212 on the upper surface 326 of the indexing disk 26 so as to rotate the indexing disk 26 one-tenth of a complete rotation. The rotation of the indexing disk 26 causes the sensor pack 122 to be rotated so that the next one of the sensor cavities 130a-j is placed in a standby position aligned with the test-sensor opening 12 formed in the housing 12. At the same time, the knife blade assembly 214 is retracted and moved towards the center of the indexing disk 26.

Next, the puller handle 28 is manually pushed inwardly from the extended position (FIG. 5a) into a testing position (FIG. 5b). The inward movement of the puller handle 28 causes the knife blade assembly 214 to pivot downwardly so that a knife blade 216 pierces a portion of the protective foil 142 covering the sensor cavity 130 in the standby position and engages the sensor 126 in the sensor cavity 130. As the puller handle 28 continues to move back towards the housing 12, the knife blade assembly 214 forces the sensor 126 out of the sensor cavity 130 and into a testing position at the testing end 35 of the housing 12. At the same time, the cam button 208 on the indexing disk drive arm 206 travels along one of the radially extending grooves 218 to prevent the indexing disk 26 from rotating.

After the sensor 126 has been completely ejected from the sensor cavity 130 and pushed into a testing position partially projecting out from the testing end 35 of the housing 12, the sensor actuator 220 engages the sensor 126 to hold the sensor 126 in the testing position and to couple the sensor 126 to the electronics assembly 222. The testing end 306 of the sensor is then inserted into a fluid sample to be tested, whereby the fluid sample is analyzed by the electronics assembly 222. The results of the analysis are then displayed on the display unit 54 of the integrated diagnostic instrument 10.

In embodiments where the fluid sample is a whole blood sample, the lancing mechanism 16 can be utilized to generate the sample. In using the lance 86 to puncture a test subject's skin, a user grasps the integrated diagnostic instrument 10 by the housing 12 and moves the slider 90 in the direction of arrow A (FIG. 2) to cock the lancing mechanism 16. The movement of the slider 90 in the direction of arrow A moves the plunger 66 in the direction of arrow A as well. This causes the spring 82 (FIG. 12) to compress. Once the spring 82 has been sufficiently compressed, a locking mechanism (not shown) prohibits the spring 82 from decompressing. A second spring (not shown) may be used to return the slider 90 to its original position. The second spring may be compressed by the slider 90 (or an extension therefrom into the housing) as the slider 90 moves in the direction of arrow A. Upon release of the slider 90, the second spring can then decompress, forcing the slider 90 back in the direction of arrow B until the slider reaches the slider dock 88.

Once the spring 82 has been compressed and locked, the user may then bring the face 102 (FIGS. 4 and 12) of the endcap 62 into contact with the skin of the test subject. The user depresses the firing button 98 to cause the locking mechanism (not shown) to release the spring 82. The spring 82 then rapidly decompresses causing the plunger 66 to move in the direction of arrow B and partially into and through the plunger opening 100 in the housing. This movement of the plunger 66 causes the lance 86 to extend, or further extend, from the endcap 62 of the lancing mechanism 16, thus, advancing the lance 86 into a test subject's skin.

During the lancing of a test subject's skin, the face 102 of the endcap 62 is placed on an area of the test subject's skin (e.g., a forearm or finger). The plunger 66 is rapidly moved in the direction of arrow B by the spring 82 to advance the lance 86 from a retracted position, wherein the lance 86 is completely contained within the endcap 62, to a lancing position, wherein the lance 86 extends through the aperture 114 of the endcap 62 and into the test subject's skin. Further movement of the lance 86 out of the endcap 62 beyond a set point may be inhibited by the plunger 66, the shaft 70, or one or more lance stop (not shown) provided within the endcap 62. The once or more lance stop may be adapted to contact the base 106 of the lance 86 as the lance 86 advances into the test subject's skin. Thus, the lancing mechanism 16 may provide uniform puncture depth for each lancing.

Once the analysis of the fluid sample is complete, the button release 32 on the upper case 22 is depressed so as to disengage the sensor actuator 220 and release the sensor 126.

### Alternative Embodiment A

An integrated diagnostic instrument for analyzing a fluid sample, comprising:

a housing having an exterior and a sensor opening formed therein;

a sensor pack having a plurality of sensor cavities, each of the plurality of sensor cavities being adapted to house a test sensor therein, the test sensor being adapted to assist in the determination of an analyte concentration in the fluid sample;

a disk drive mechanism disposed in the housing and moveable between a standby position, an extended position, and a testing position, the disk drive mechanism removing a test sensor from the sensor pack and partially ejecting the test sensor through the sensor opening of the housing as the disk drive mechanism is moved between positions; and

a lancing mechanism having
(i) a lance holder adapted to removably engages a base of a lance,
(ii) a plunger coupled to the lance holder, the plunger having a central portion,
(iii) a shaft running through the central portion of the plunger, the plunger being adapted to move along the shaft, the shaft having an end portion that is adapted to secure the shaft to the integrated diagnostic instrument,
(iv) a spring at least partially surrounding the shaft, the spring being located between the plunger and the end portion of the shaft, and
(v) a slider located on a rail on the exterior of the housing, the slider being adapted to move along the rail in a first direction to compress the spring and wherein the decompressing of the spring causes the plunger and lance holder to rapidly move in a second direction opposite the first direction

### Alternative Embodiment B

The integrated diagnostic instrument of Alternative Embodiment A, the lancing mechanism further having a firing button located on a slider dock, the firing button being adapted to allow the spring to rapidly decompress when the firing button is depressed.

### Alternative Embodiment C

The integrated diagnostic instrument of Alternative Embodiment A, the lancing mechanism further having an endcap that covers the plunger, the endcap being adapted to regulate the distance the spring can cause the plunger and lance holder to move in the second direction.

### Alternative Embodiment D

The integrated diagnostic instrument of Alternative Embodiment C, wherein the endcap is removably attached to the housing.

### Alternative Embodiment E

The integrated diagnostic instrument of Alternative Embodiment A, wherein the disk drive mechanism removes the test sensor from the sensor pack and partially ejects the test sensor through the sensor opening as the disk drive mechanism is moved from the extended position to the testing position.

### Alternative Embodiment F

The integrated diagnostic instrument of Alternative Embodiment A, wherein the sensor pack is substantially circular.

### Alternative Embodiment G

The integrated diagnostic instrument of Alternative Embodiment A, wherein the test sensors are stored within the sensor cavities in the sensor pack by enclosing the sensor cavities with foil.

### Alternative Embodiment H

The integrated diagnostic instrument of Alternative Embodiment A, the test sensor being adapted to electrochemically assist in the determination of an analyte concentration in the fluid sample.

### Alternative Embodiment I

The integrated diagnostic instrument of Alternative Embodiment A, wherein the lancing mechanism is offset from the sensor opening by at least 20 degrees.

### Alternative Process J

A method for collecting and analyzing a concentration of an analyte in a fluid sample, comprising the acts of:

mounting a sensor pack on an indexing disk within a housing of an integrated diagnostic instrument, the sensor pack having a plurality of sensor cavities each being adapted to house a test sensor therein, the test sensor being adapted to assist in the determination of an analyte concentration in the fluid sample;

actuating a disk drive mechanism to remove a test sensor from the sensor pack and partially eject the test sensor through a sensor opening of the housing;

lancing the skin of a test subject with a lancing mechanism to obtain a fluid sample, the lancing mechanism at least partially contained within the housing of the integrated diagnostic instrument, the integrated diagnostic instrument being in a first position when lancing;

moving the integrated diagnostic instrument from the first position to a second position;

applying the obtained fluid sample from the test subject to the partially ejected test sensor, the integrated diagnostic instrument being in the second position when applying the obtained fluid sample; and

determining the analyte concentration of the fluid sample.

### Alternative Process K

The method of Alternative Process J, wherein the lancing of the skin includes
(i) moving a slider in a first direction, the movement of the slider causing a plunger to move in the first direction and compress a spring, and
(ii) depressing a firing button causing the spring to decompress and move the plunger in a second direction, opposite the first direction.

### Alternative Process L

The method of Alternative Process J, wherein the fluid sample is a whole blood sample.

### Alternative Process M

The method of Alternative Process J, wherein the analyte is glucose in a whole blood sample.

### Alternative Process N

The method of Alternative Process J, wherein the sensor pack is mounted on the indexing disk by pivoting a lower case relative to an upper case to access the indexing disk, the lower case and the upper case form the housing.

### Alternative Process O

The method of Alternative Process J, wherein a substantially circular sensor pack is mounted on the indexing disk.

### Alternative Process P

The method of Alternative Process J, wherein the determination of the analyte concentration in the fluid sample is performed through an electrochemical analysis of the fluid sample.

### Alternative Process Q

The method of Alternative Process J, wherein the integrated diagnostic instrument is moved at least 20 degrees from the first position to the second position.

### Alternative Process R

The method of Alternative Process J, wherein the integrated diagnostic instrument is moved at least 45 degrees from the first position to the second position.

While the invention is susceptible to various modifications and alternative forms, specific embodiments and methods thereof have been shown by way of example in the drawings and are described in detail herein. It should be understood, however, that it is not intended to limit the invention to the particular forms or methods disclosed, but, to the contrary, the intention is to cover all modifications, equivalents and alternatives falling within the spirit and scope of the invention as defined by the appended claims.

1. An integrated diagnostic instrument for analyzing a fluid sample, comprising:
a housing having an exterior and a sensor opening formed therein;
a sensor pack having a plurality of sensor cavities, each of the plurality of sensor cavities being adapted to house a test sensor therein, the test sensor being adapted to assist in the determination of an analyte concentration in the fluid sample;
a disk drive mechanism disposed in the housing and moveable between a standby position, an extended position, and a testing position, the disk drive mechanism removing a test sensor from the sensor pack and partially ejecting the test sensor through the sensor opening of the housing as the disk drive mechanism is moved between positions; and
a lancing mechanism having
(i) a lance holder adapted to removably engages a base of a lance,
(ii) a plunger coupled to the lance holder, the plunger having a central portion,
(iii) a shaft running through the central portion of the plunger, the plunger being adapted to move along the shaft, the shaft having an end portion that is adapted to secure the shaft to the integrated diagnostic instrument,
(iv) a spring at least partially surrounding the shaft, the spring being located between the plunger and the end portion of the shaft, and
(v) a slider located on a rail on the exterior of the housing, the slider being adapted to move along the rail in a first direction to compress the spring and wherein the decompressing of the spring causes the plunger and lance holder to rapidly move in a second direction opposite the first direction.

2. The integrated diagnostic instrument of claim 1, the lancing mechanism further having a firing button located on a slider dock, the firing button being adapted to allow the spring to rapidly decompress when the firing button is depressed.

3. The integrated diagnostic instrument of claim 1, the lancing mechanism further having an endcap that covers the plunger, the endcap being adapted to regulate the distance the spring can cause the plunger and lance holder to move in the second direction.

4. The integrated diagnostic instrument of claim 3, wherein the endcap is removably attached to the housing.

5. The integrated diagnostic instrument of claim 1, wherein the disk drive mechanism removes the test sensor from the sensor pack and partially ejects the test sensor through the sensor opening as the disk drive mechanism is moved from the extended position to the testing position.

6. The integrated diagnostic instrument of claim 1, wherein the sensor pack is substantially circular.

7. The integrated diagnostic instrument of claim 1, wherein the test sensors are stored within the sensor cavities in the sensor pack by enclosing the sensor cavities with foil.

8. The integrated diagnostic instrument of claim 1, the test sensor being adapted to electrochemically assist in the determination of an analyte concentration in the fluid sample.

9. The integrated diagnostic instrument of claim 1, wherein the lancing mechanism is offset from the sensor opening by at least 20 degrees.

10. A method for collecting and analyzing a concentration of an analyte in a fluid sample, comprising the acts of:
mounting a sensor pack on an indexing disk within a housing of an integrated diagnostic instrument, the sensor pack having a plurality of sensor cavities each being adapted to house a test sensor therein, the test sensor being adapted to assist in the determination of an analyte concentration in the fluid sample;
actuating a disk drive mechanism to remove a test sensor from the sensor pack and partially eject the test sensor through a sensor opening of the housing;
lancing the skin of a test subject with a lancing mechanism to obtain a fluid sample, the lancing mechanism at least partially contained within the housing of the integrated diagnostic instrument, the integrated diagnostic instrument being in a first position when lancing;
moving the integrated diagnostic instrument from the first position to a second position;
applying the obtained fluid sample from the test subject to the partially ejected test sensor, the integrated diagnostic instrument being in the second position when applying the obtained fluid sample; and
determining the analyte concentration of the fluid sample.

11. The method of claim 10, wherein the lancing of the skin includes
moving a slider in a first direction, the movement of the slider causing a plunger to move in the first direction and compress a spring, and
depressing a firing button causing the spring to decompress and move the plunger in a second direction, opposite the first direction.

12. The method of claim 10, wherein the fluid sample is a whole blood sample.

13. The method of claim 10, wherein the analyte is glucose in a whole blood sample.

14. The method of claim 10, wherein the sensor pack is mounted on the indexing disk by pivoting a lower case relative to an upper case to access the indexing disk, the lower case and the upper case form the housing.

15. The method of claim 10, wherein a substantially circular sensor pack is mounted on the indexing disk.

16. The method of claim 10, wherein the determination of the analyte concentration in the fluid sample is performed through an electrochemical analysis of the fluid sample.

17. The method of claim 10, wherein the integrated diagnostic instrument is moved at least 20 degrees from the first position to the second position.

18. The method of claim 10, wherein the integrated diagnostic instrument is moved at least 45 degrees from the first position to the second position.
